(19) United States
(12) Patent Application Publication
(10) Pub. No.: US 2022/0409116 A1
(43) Pub. Date: Dec. 29, 2022

(54) Electromyography Needle Electrode Having an Enhanced Ease of Insertion

(71) Applicant: Cadwell Laboratories, Inc., Kennewick, WA (US)

(72) Inventor: John A. Cadwell, Richland, WA (US)

(21) Appl. No.: 17/809,319

(22) Filed: Jun. 28, 2022

Related U.S. Application Data
(60) Provisional application No. 63/215,547, filed on Jun. 28, 2021.

(51) Int. Cl. A61B 5/262; A61B 5/296; A61B 5/271; A61B 5/263

(52) U.S. Cl. CPC A61B 5/262; A61B 5/296; A61B 5/271; A61B 5/263; A61B 2562/125

(57) ABSTRACT

An electrode configured for use in electromyography procedures including a shaft having a first end and a second end, where the shaft consists of a conductive material; an electrically insulative first coating configured to encase the conductive material; a tapered tip at the first end of the shaft, where the tip is angled and is formed by removing the first coating from a first portion of the shaft and exposing a first length of conductive material; and a hub positioned at the second end of the shaft, wherein the hub is positioned after removing the first coating from a second portion of the shaft and exposing a second length of conductive material, wherein the hub is configured to electrically couple a lead wire to the second length of conductive material at the second end.

### CROSS-REFERENCE

The present application relies on, for priority, U.S. Patent Provisional Application No. 63/215,547, entitled “Electromyography Needle Electrode Having an Enhanced Ease of Insertion” and filed on Jun. 28, 2021, which is hereby incorporated by reference in its entirety.

### FIELD

The present specification is related generally to the field of medical devices. More specifically the present specification is related to an improved needle electrode that has a reduced insertion force, thereby enabling an improved approach to measuring electrical activity in a patient during an electromyography.

### BACKGROUND

Electromyography (EMG) is a low-risk invasive procedure in which a small, insulated needle with an exposed tip, having a known exposed surface area, is inserted through the skin into muscle and is used to record muscle electrical activity of a patient. The needle is incrementally moved within each muscle both axially (in and out) and radially (side to side).

Certain physical parameters of EMG electrode needles are critically important. EMG needles need to be of a sufficient length to reach the muscles being evaluated and, additionally, need to have a minimum level of stiffness to allow for accurate placement. For a longer needle to have the stiffness needed for accurate placement, it needs to have a larger diameter. However, patients find larger diameter needles to create greater discomfort during insertion. Therefore, this is a need to find a better balance between needle stiffness and its dimensions.

EMG needles have two common forms: monopolar and concentric. Monopolar needles have a single conductor surrounded by an insulator and an exposed tip. Concentric needles have a central conductor, an insulating layer, and an outer conducting layer that extends nearly to the tip. Concentric needles typically have one or more additional conductive layers and two lead attachments. Needles with multiple contacts also exist.

Furthermore, EMG needles are typically insulated with a coating. However, since the insulated needle is inserted and removed through the skin multiple times to examine multiple muscles, that insulative coating can be damaged during insertion and when the needle flexes. Damage to the insulative coating compromises test results and require use of a fresh, intact needle. To minimize insulative coating erosion, improved EMG needle coatings are required. Currently, EMG needle electrodes are coated using polytetrafluoroethylene (PTFE) or perylene. Both of these materials are relatively soft, pliable and moderately adherent. However, they have an innate friction that affects insertion force and the coatings are often thick to provide the requisite mechanical strength.

Accordingly, there is need for a low friction, sharp, durable EMG needle that improves patient comfort while enabling clinician with easier, more accurate placement and high signal quality. Furthermore, there is a need for an EMG electrode needle that better balances its dimensions and stiffness properties. Finally, there is a need for materials and approaches to coating EMG needles that result in insulative coatings which, relative to conventional coatings, are less susceptible to erosion, require less insertion force, and provide the requisite mechanical strength without being too thick.

### SUMMARY

The following embodiments and aspects thereof are described and illustrated in conjunction with systems, tools and methods, which are meant to be exemplary and illustrative, not limiting in scope. The present application discloses numerous embodiments.

In some embodiments, the present specification is directed towards an electrode configured for use for use in electromyography procedures, comprising: a cylindrical shaft having a first end and a second end, wherein the cylindrical shaft consists of a conductive material; a first coating that is electrically insulative and is configured to encase an entirety of the conductive material; a tapered tip defined by an angled surface and positioned at the first end of the shaft, wherein the tip comprises a portion of the cylindrical shaft having a first portion of the first coating removed therefrom to thereby expose a first length of conductive material that was positioned under the removed first portion of first coating and form the angled surface; and a hub positioned at the second end of the shaft, wherein the hub is positioned on a portion of the cylindrical shaft having a second portion of the first coating removed therefrom to thereby expose a second length of conductive material that was positioned under the removed second portion of first coating, and wherein the hub is configured to electrically couple a lead wire to the second length of conductive material at the second end.

Optionally, the conductive material comprises tungsten or stainless steel.

Optionally, the conductive material has a thickness ranging from 25 to 32 gauge.

Optionally, the first coating has a minimum thickness of 20 microns.

Optionally, the first coating comprises at least one biocompatible diamond-like carbon (DLC) material.

Optionally, the tapered tip comprises at least two bevels.

Optionally, the cylindrical shaft and tapered tip has a first portion and a second portion, wherein the first portion is of a first length and has a substantially uniform diameter, wherein the second portion is of a second length and has a tapered surface, and wherein the tip comprises the second portion. Optionally, the electrode further comprises a second coating positioned over the first coating, wherein the second coating is at least one of molybdenum disulfide, tungsten disulfide or silicone oil. Optionally, the electrode further comprises a conductive third coating positioned over the second coating, wherein the third coating comprises a doped diamond-like carbon material.

In some embodiments, the present specification is directed toward a method of fabricating an electrode in a shape of a needle and configured for use in electromyography procedures, comprising: acquiring a conductive length of wire; deforming the length of wire at a plurality of predetermined intervals, wherein said deforming reduces a diameter of the length of wire at each of the plurality of predetermined intervals and generates a plurality of deformations; placing the length of wire on a support; applying an insulative first coating to the length of wire to obtain a coated length of wire; cutting the coated length of wire at each of the plurality of deformations to obtain a plurality of coated shafts, wherein each of the plurality of coated shafts is defined by a first end and a second end; grinding the first end of each of the plurality of coated shafts to transform a cylindrical first end into an angled tip with at least two bevels; grinding the second end of each of the plurality of coated shafts to remove a portion of the first coating at the second end and thereby expose a conductive length of wire under the removed portion of the first coating; positioning a hub at the second end of each of the plurality of coated shafts; electrically connecting a lead wire to the exposed conductive length of wire at the second end of each of the plurality of coated shafts via the hub; and applying a second coating to each of the plurality of coated shafts and tips, wherein the second coating is different than the first coating.

Optionally, the method further comprises cleaning the length of wire after deforming the length of wire at the plurality of predetermined intervals.

Optionally, the method further comprises stretching the length of wire beyond its elastic limit.

Optionally, the insulative first coating is applied using a vacuum plasma deposition process, wherein the vacuum plasma deposition process is repeated to achieve a thickness of the first coating of at least 20 microns.

Optionally, the thickness of the first coating reduces an electrical capacitance of the electrode.

Optionally, the first coating is a biocompatible diamond-like carbon (DLC) material.

Optionally, the second coating comprises at least one of molybdenum disulfide, tungsten disulfide or silicone oil.

Optionally, each of the plurality of coated shafts has a first portion and a second portion, wherein the first portion is of a first length and has a substantially uniform diameter, wherein the second portion is of a second length and has a tapered surface, and wherein the second portion comprises the tip at the first end of each of the plurality coated shafts.

Optionally, the length of wire comprises at least one of tungsten or stainless steel.

Optionally, the length of wire has a thickness ranging from 25 to 32 gauge.

In some embodiments, the present specification discloses a needle electrode for use in electromyography procedures, comprising: a shaft having a first end and a second end, wherein the shaft includes a conductive core that is covered by an electrically insulative first coating followed by a second coating, wherein the first coating is of at least one DLC material, wherein the second coating is of a lubricant, and wherein the first coating has a thickness of at least 20 microns; a tip at the first end, wherein the tip is formed by grinding the first end to expose the underlying conductive core, and wherein the tip includes at least two bevels; and a hub positioned at the second end, wherein the hub is positioned after grinding the second end to expose the underlying conductive core, and wherein the exposed conductive core at the second end is electrically coupled to a lead wire via the hub.

Optionally, the shaft has a first portion and a second portion, wherein the first portion is of a first length and has a substantially uniform diameter, wherein the second portion is of a second length and has a tapering surface, and wherein the second portion includes the tip at the first end.

In some other embodiments, the present specification discloses a needle electrode for use in electromyography procedures, comprising: a shaft having a first end and a second end, wherein the shaft comprises a conductive core; an electrically insulative first coating that encases the conductive core; a tip at the first end, wherein the tip is formed by removing the first coating at the first end to expose the underlying conductive core; and a hub positioned at the second end, wherein the hub is positioned after removing the first coating at the second end to expose the underlying conductive core, and wherein the exposed conductive core at the second end is electrically coupled to a lead wire.

Optionally, the conductive core comprises tungsten or stainless steel.

Optionally, the conductive core has a thickness ranging from 25 to 32 gauge.

Optionally, the first coating has a thickness of at least 20 microns.

Optionally, the first coating comprises at least one biocompatible diamond-like carbon (DLC) material.

Optionally, the tip comprises at least two bevels.

Optionally, the shaft has a first portion and a second portion, wherein the first portion is of a first length and has a substantially uniform diameter, wherein the second portion is of a second length and has a tapered surface, and wherein the second portion includes the tip at the first end. Optionally, the needle electrode further comprises a second coating over the first coating, wherein the second coating is at least one of molybdenum disulfide, tungsten disulfide or silicone oil. Optionally, the needle electrode further comprises a third coating over the second coating. Optionally, the needle electrode further comprises: a conductive second coating applied over the first coating; and a conductive third coating applied over the second coating, wherein the third coating comprises a doped DLC material.

In still other embodiments, the present specification also discloses a method of fabricating a needle electrode for use in electromyography procedures, comprising: acquiring a conductive strand of wire; deforming the strand of wire at a plurality of predetermined intervals, wherein said deforming reduces a diameter of the strand of wire at said plurality of predetermined intervals; cleaning the strand of wire; placing the strand of wire on a coating rack; applying an insulative first coating to the strand of wire to obtain a coated strand of wire; cutting the coated strand of wire at said plurality of predetermined intervals to obtain a plurality of coated needle shafts, wherein each of the plurality of coated needle shafts is of a predetermined length; grinding a first end of each of the plurality of coated needle shafts to generate a tip with at least two bevels; grinding a second end of each of the plurality of coated needle shafts to expose the underlying conductive strand of wire; positioning a hub at the second end of each of the plurality of coated needle shafts; electrically connecting a lead wire to the exposed conductive strand of wire at the second end of each of the plurality of coated needle shafts; and applying a second coating to each of the plurality of coated needle shafts.

Optionally, the strand of wire is stretched longitudinally past its elastic limit.

Optionally, the insulative first coating is applied using a vacuum plasma deposition process, wherein the vacuum plasma deposition process is repeated to achieve a thickness of the first coating of at least 20 microns.

Optionally, a thickness of the first coating reduces an electrical capacitance of the needle electrode.

Optionally, the first coating is of at least one biocompatible diamond-like carbon (DLC) material.

Optionally, the second coating comprises at least one of molybdenum disulfide, tungsten disulfide or silicone oil.

Optionally, each of the plurality of coated needle shaft has a first portion and a second portion, wherein the first portion is of a first length and has a substantially uniform diameter, wherein the second portion is of a second length and has a tapered surface, and wherein the second portion includes the tip at the first end.

Optionally, the strand of wire is of tungsten or stainless steel.

Optionally, the strand of wire has a thickness ranging from 25 to 32 gauge.

In still other embodiments, the present specification also discloses a needle electrode for use in electromyography procedures, comprising: a shaft having a first end and a second end, wherein the shaft includes a conductive core that is covered by an electrically insulative first coating followed by a second coating, wherein the first coating is of at least one DLC material, wherein the second coating is of a lubricant, and wherein the first coating has a thickness of at least 20 microns; a tip at the first end, wherein the tip is formed by grinding the first end to expose the underlying conductive core, and wherein the tip includes at least two bevels; and a hub positioned at the second end, wherein the hub is positioned after grinding the second end to expose the underlying conductive core, and wherein the exposed conductive core at the second end is electrically coupled to a lead wire.

Optionally, the shaft has a first portion and a second portion, wherein the first portion is of a first length and has a substantially uniform diameter, wherein the second portion is of a second length and has a tapering surface, and wherein the second portion includes the tip at the first end.

The aforementioned and other embodiments of the present specification shall be described in greater depth in the drawings and detailed description provided below.

### BRIEF DESCRIPTION OF THE DRAWINGS

These and other features and advantages of the present specification will be appreciated, as they become better understood by reference to the following detailed description when considered in connection with the accompanying drawings, wherein:

FIG. 1A is a longitudinal cross-sectional view of a needle electrode for measurement of electrical activity of a patient's muscles during electromyography (EMG), in accordance with some embodiments of the present specification;

FIG. 1B is a side elevation view showing a first transverse cross-section of a needle with a single coating in accordance with some embodiments of the present specification;

FIG. 1C is a side elevation view showing a transverse cross-section of a needle electrode with two coatings, in accordance with some embodiments of the present specification;

FIG. 1D is a side elevation view showing a transverse cross-section of a needle with multiple coatings in accordance with some embodiments of the present specification;

FIG. 1E is a side elevation view showing a transverse cross-section of a needle electrode with multiple coatings including a conductive outer coating, in accordance with some embodiments of the present specification;

FIG. 2 is a schematic of a plurality of needle electrodes with associated lead wires, in accordance with some embodiments of the present specification;

FIG. 3A is a flow chart depicting a plurality of exemplary steps of a method of fabricating a needle electrode for use in an EMG procedure, in accordance with some embodiments of the present specification;

FIG. 3B is a pictorial illustration of the plurality of exemplary steps of the method of FIG. 3A, in accordance with some embodiments of the present specification; and

FIG. 4 is a flow chart depicting a plurality of exemplary steps of a method of fabricating a Botox injection needle, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “comprise” “include” and “have”, and forms thereof, are not necessarily limited to members in a list with which the words may be associated. It should be noted herein that any feature or component described in association with a specific embodiment may be used and implemented with any other embodiment unless clearly indicated otherwise.

As used herein, the indefinite articles “a” and “an” mean “at least one” or “one or more” unless the context clearly dictates otherwise.

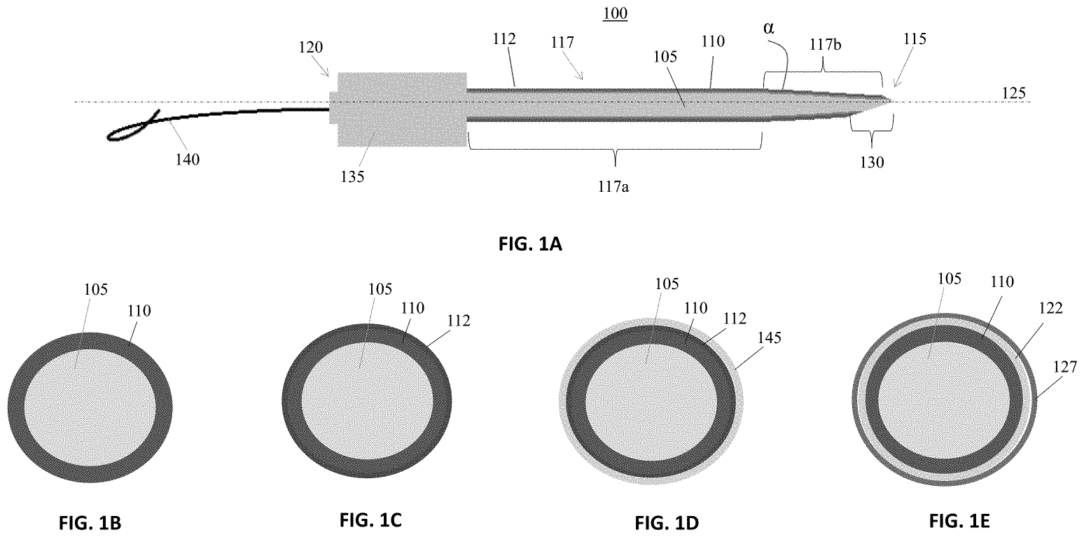

FIG. 1A illustrates a longitudinal cross-sectional view of a needle electrode 100 for measurement of electrical activity of a patient's muscles during electromyography (EMG), in accordance with some embodiments of the present specification. In some embodiments, as shown in a first transverse cross-sectional view of FIG. 1B, the needle electrode 100 includes a conductive inner core 105 that is surrounded, encased or covered by a non-conductive or electrically insulative first coating or sheath 110. In some embodiments, the inner core 105 is a wire of a conductive and stiff material such as, but not limited to, tungsten or stainless steel. In some embodiments, a thickness of the wire ranges from 25 to 32 gauge.

As shown in FIG. 1A, the needle electrode 100 comprises a shaft 117 having a first distal end 115, a second proximal end 120 and a longitudinal axis 125. The electrically insulative first coating or sheath 110 is removed (such as, for example, by grinding) at the first end 115 to expose the conductive inner core 105 and form a tip 130. The second proximal end 120 includes a hub 135, used to manually hold and manipulate the needle, and through which at least one lead wire 140 is electrically coupled to the needle electrode 100. In embodiments the lead wire 140 is used to carry the electrical signal generated by a patient's muscle to the amplifier of an EMG system for display.

In some embodiments, the tip 130 has a length (along the longitudinal axis 125) ranging from 0.1 mm to 1 mm. In various embodiments, the needle electrode 100 is characterized by various properties, which are described in detail below.

The insulative first coating or sheath 110 is thin in order to allow for the overall needle to be thin to reduce discomfort. In some embodiments, the electrically insulative first coating or sheath 110 has a thickness ranging from 1 micron to 50 microns, and preferably on the order of approximately 20 microns. In embodiments, a thickness of approximately 20 microns is further desired to reduce undesired signal coupling along the shaft 117 of the needle due to the capacitance of the needle 100. As is known, capacitance is equal to permittivity×area/distance. Therefore capacitance drops as thickness of the insulative coating or sheath 110 on the needle 100 increases. In different embodiments, a diameter of the shaft 117 varies based on length of the needle 100.

The insulative first coating or sheath 110 is fabricated from a material that is both tough and highly adherent to the conductive inner core 105, to substantially reduce damage to the first coating or sheath 110 during use. In addition, the insulative first coating or sheath 110 is fabricated using a material having high lubricity to reduce patient discomfort. Accordingly, in some embodiments, the electrically insulative first coating or sheath 110 is a biocompatible diamond-like carbon (DLC) material containing significant amounts of sp³ hybridized carbon atoms. In embodiments, the first coating or sheath 110 is fabricated from materials that provide lubricity such as, but not limited to, fluorinated DLC compounds, Molybdenum Disulfide or Tungsten Disulfide. In some embodiments, the materials are used to coat the needle 100 sequentially in order to obtain optimal electrical, mechanical and frictional properties, and to produce a second category of needles (concentric) used in EMG. In an embodiment, a concentric needle comprises an inner conductor core, a first insulating layer, and a second outer conductive layer connected to a second electrical lead wire used as an electrical reference. Referring to FIG. 1, in embodiments, additives are mixed with the DLC materials used for coating the needle 100 for improving needle properties such as, but not limited to, hardness, conductivity, coefficient of friction, and antimicrobial action. For example, adding nitrogen to DLC materials used for coating increases conductivity of the needle, while hydrogenated DLC and fluorinated DLC reduce friction of the needle.

In embodiments, the needle 100 requires a low insertion force for penetrating tissue, as the needle 100 has a low surface friction enabling it to penetrate a patient's skin easily. Accordingly, in some embodiments, as shown in a second transverse cross-sectional view of FIG. 1C, the needle electrode 100 further includes a lubricious second coating 112 over the insulative first coating or sheath 110. In some embodiments, thickness of the second coating 112 ranges from 2 microns to 4 microns. The lubricious second coating 112 reduces the coefficient of friction of the needle electrode 100 by up to 50%. The friction may otherwise result in dimpling of the skin on insertion of needle 100, and lifting of the skin upon retraction of needle 100. The lubricious second coating 112 causes the needle shaft 117 and tip 130 (FIG. 1A) to have a slippery surface resulting in a more precise control of the needle electrode 100, increased patient comfort, reduced insertion force, reduced tissue irritation/damage and ease-of-use. Accordingly, in some embodiments, the second coating 112 is of a dry lubricant such as, but not limited to, molybdenum disulfide, tungsten disulfide or a liquid lubricant such as, but not limited to, silicone oil (which is a liquid polymerized siloxane with organic side chains).

As specified above, a stiffness of the needle is required to increase with an increase in its length and/or thickness. In embodiments, the needle electrode 100 of the present specification has a higher overall stiffness compared to an overall diameter of the coated shaft 117. Relative to conventional needles, in embodiments, the needle 100 has a predefined stiffness associated with a required shaft diameter, wherein the predefined stiffness range is less than conventional needle stiffness ranges. In an embodiment, for any given material, the stiffness is related to the third (3rd) power of the diameter (D). Hence, in an embodiment, a stiffer material (such as, but not limited to Tungsten) may be used to construct the needle substrate or inner core 105 of the needle electrode 100 allowing for smaller gauge needles to have a predefined/required stiffness.

In embodiments, the needle electrode comprises a cantilevered shaft 117 that comprises a tapered portion so that a diameter of the tip 130 is smaller than that of a base of the shaft 117. Accordingly, in some embodiments (as shown in FIG. 1A), the shaft 117 has a base or first portion 117a of length ‘Lₐ’ having a substantially uniform diameter ‘Dₐ’ (the surface of the base or first portion 117a being substantially parallel to the longitudinal axis 125) and a second portion 117b of length ‘L_{b}’ having a predefined tapered profile that culminates into the tip 130 at the first end 115. When moving from the base or first portion 117a to the tip 130, the second portion 117b has an incrementally diminished or reduced diameter along its length ‘L_{b}’. The base or first portion 117a supports the hub 135 at the second proximal end 120. In some embodiments, the surface of the second portion 117b makes an angle ‘α’ with the longitudinal axis 125. In various embodiments, the angle ‘α’ ranges from 10 degrees to 20 degrees.

In embodiments, the needle electrode 100 has a plurality of bevels ground on the tip 130 for sharpness. Bevels expose cutting edges at tip 130 of the needle 100. The bevels also aid to expose a precise and repeatable recording surface area at the tip 130. In some embodiments, the tip 130 has preferably two bevels and more preferably three bevels. Three bevels come together to form a sharp tip. In some embodiments, one of the three bevels is on a front face, and the remaining two bevels are angled like the hull of a boat, on a back face (opposite to the front face).

It should be appreciated that, in various embodiments, the needle electrode 100 having at least one DLC material as the first coating or sheath 110 in conjunction with a dry or liquid lubricant as the second coating 112 provides significant improvements (over prior art needle electrodes) in at least the following properties: lubricity, signal quality, cleaner insertion/removal, thin coatings, durability, sharp edge retention, electrically insulative, needle smoothness, well defined exposed surface area of the tip 130, biocompatibility, good adhesion to the conductive inner core 105, corrosion resistance, and/or anti-fouling.

In some embodiments, as shown in a third transverse cross-sectional view of FIG. 1D, the needle electrode 100 includes the conductive inner core 105, the electrically insulative first coating or sheath 110 covering the inner core 105, the lubricious second coating 112 applied over the first coating or sheath 110, followed by a lubricious third coating 145 that is applied over the second coating 112. In embodiments, the lubricious coating 145 is added to reduce drag as the needle 100 is inserted through skin, in order to reduce patient discomfort. In an embodiment, the second coating is vapor deposited. In an embodiment, the second coating is molybdenum disulfide. In embodiments, the third coating may be silicone oil that is deposited directly or by any other method that is known by those of ordinary skill in the art.

While in some embodiments the needle electrode 100 is fabricated as a monopolar needle (as shown in the cross-sectional views of FIGS. 1B, 1C and 1D), in some embodiments the needle electrode 100 is fabricated as a concentric needle. Referring now to FIG. 1E, in this embodiment, the needle electrode 100 is fabricated as a concentric needle having the conductive inner core 105, the electrically insulative first coating or sheath 110 covering the inner core 105, a conductive second coating or metallization layer 122 applied over the first coating 110, followed by a third coating or layer 127 of conductive but low friction material such as, but not limited to, doped DLC (for example, nitrogen doped DLC). In embodiments, the metallization layer 122 is made of materials such as but not limited to, gold, nickel, and numerous alloys. The overall diameter of the needle electrode 100 when configured as a concentric needle is smaller compared to prior art concentric needle electrodes.

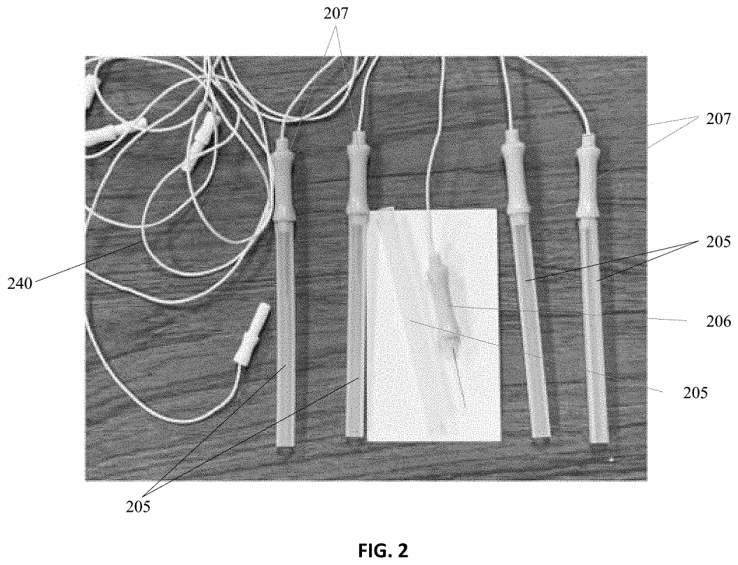

When fabricated as a monopolar needle, the needle electrode 100 includes a single lead wire 140. FIG. 2 shows a single monopolar needle electrode 206 outside of a removable protective covering 205, and a plurality of monopolar needle electrodes 207 within their protective coverings 205. Each of the monopolar needle electrodes 206, 207 are attached to their respective single lead wires 240. Referring to FIGS. 1 and 2, however, when fabricated as a concentric needle, the needle electrode 100 comprises a first lead wire 140 electrically coupled to the inner core 105, that acts as an active electrode, and a second lead wire (not shown in FIG. 1) electrically coupled to the conductive second coating or metallization layer 122 that acts as a reference electrode.

### Method of Fabricating a Needle Electrode

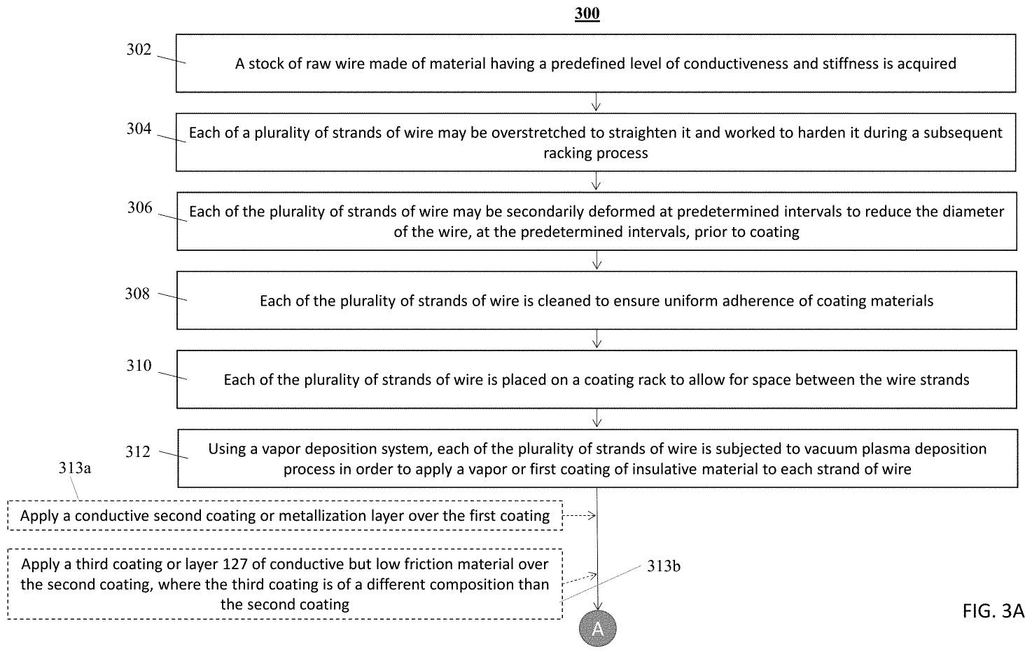

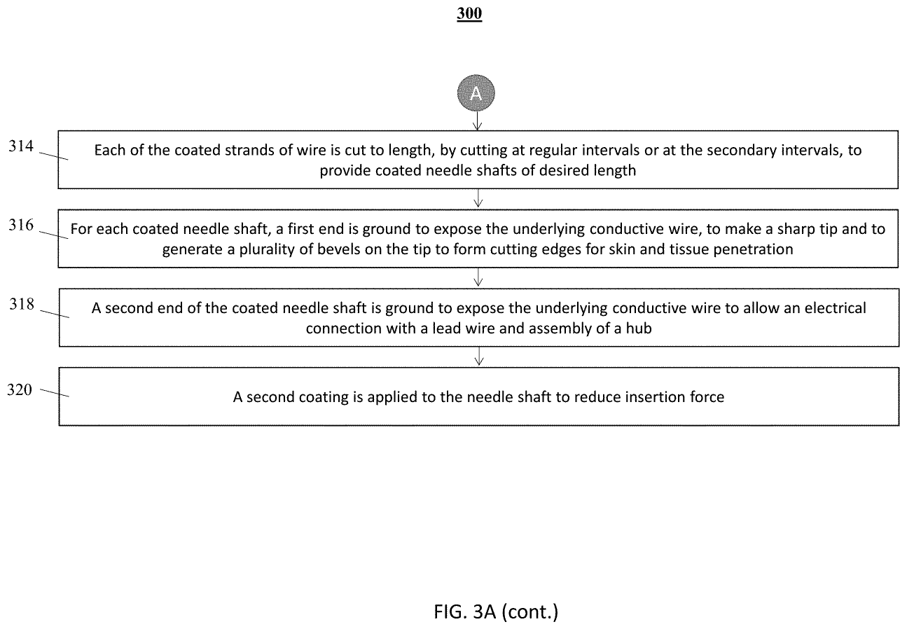

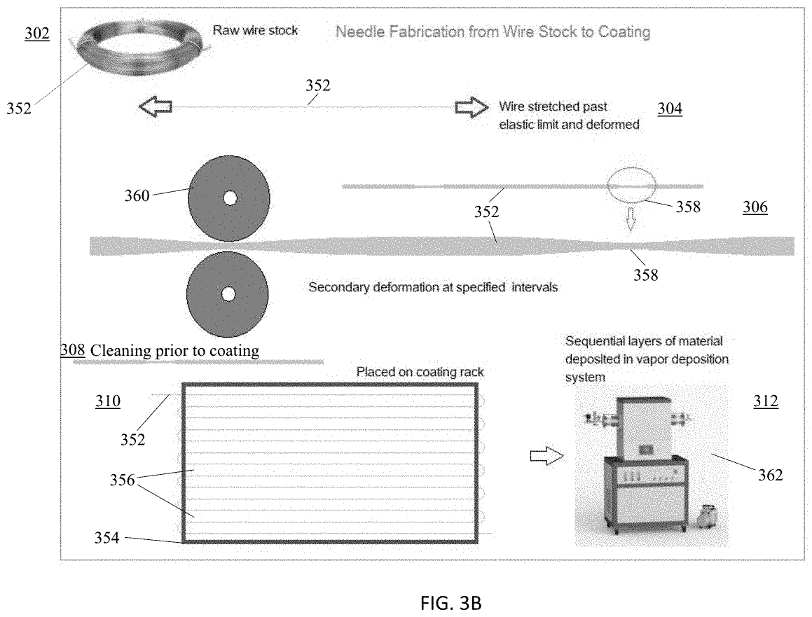

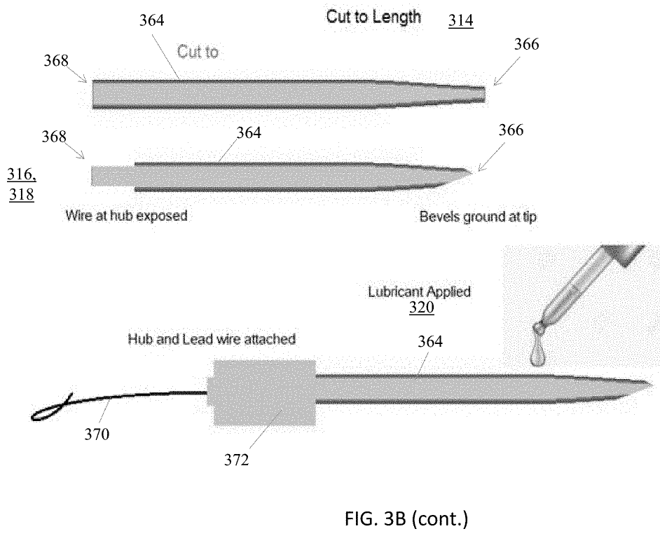

FIG. 3A is a flow chart and FIG. 3B is a pictorial illustration of a plurality of exemplary steps of a method 300 of fabricating a needle electrode for use in EMG procedures, in accordance with some embodiments of the present specification. Referring now to FIGS. 3A and 3B, at step 302, a stock of a plurality of strands of wire 352 of a conductive and stiff material is acquired. In some embodiments, the wire 352 is made of materials such as, but not limited to, tungsten or stainless steel having high stiffness. In some embodiments, a preferred thickness of the wire 352 ranges from 25 gauge to 32 gauge.

Optionally, at step 304, each of the plurality of strands of wire 352 may be overstretched (that is, slightly deformed by stretching the wire 352 longitudinally past its elastic limit) to straighten said strands and worked to harden the straightened strands during a subsequent racking process.

Optionally, at step 306, each of the plurality of strands of wire 352 may be secondarily deformed at predetermined intervals 358 corresponding to a desired needle length (for example, every 3 to 8 cm) in order to reduce the diameter of the wire 352, at the predetermined intervals 358, prior to coating. In some embodiments, the reduction of the diameter of the wire 352 (at the predetermined intervals 358) is accomplished by passing the wire through rollers 360. In some embodiments, the reduced diameter of the wire 352, at the predetermined intervals 358, enables generation of a needle shaft having a base or first portion with a substantially uniform diameter and a second portion with a predefined taper that culminates into a tip.

At step 308, each of the plurality of strands of wire 352 is cleaned prior to coating, in subsequent steps, in order to ensure uniform adherence of coating materials. At step 310, each of the plurality of strands of wire 352 is placed on a coating rack 354 to allow for space 356 between the turns of each of the plurality of wire strands. In embodiments, the rack 354 is sized to fit in commercially available vapor deposition equipment (for vacuum plasma deposition). The spacing 356 is needed for vapors to reach the full circumference of each wire strand 352. Each wire strand provides a yield of a plurality of needles. In some embodiments, each wire strand provides or yields 20 to 50 needles, for example.

At step 312, using a vapor deposition system 362, each of the plurality of strands of wire 352 is subjected to a vacuum plasma deposition process (such as, for example, an RF-based plasma-assisted chemical vapor deposition (PACVD)) in order to apply a vapor or first coating of insulative material to each strand of wire 352. In some embodiments, the vapor deposition process provides the first coating of a thickness of at least 20 microns. It should be appreciated that the vapor deposition process continues for 10 to 20 times the coating time needed for most prior art applications in order to achieve the at least 20 microns thick first coating. The first coating is thicker, compared to prior art applications, in order to reduce the electrical capacitance of the needle body.

In various embodiments, the electrically insulative first coating is of at least one biocompatible Diamond-like Carbon (DLC) material such as, but not limited to, Fluorinated DLC compounds having high lubricity. In some embodiments, the first coating may comprise one or more of the DLC materials, applied sequentially to obtain optimal electrical, mechanical and frictional properties. In some embodiments, the first coating may include one or more of the following materials applied sequentially to obtain optimal electrical, mechanical and frictional properties: DLC materials, titanium nitride (TiN) coating, TiNAg which is antimicrobial, ceramic-like/epoxy/polyurethane (Polysiloxane), aluminum titanium nitride (Altin) and silver/silver chloride.

At step 314, each of the coated strands of wire is cut to length, by cutting at regular intervals or at the secondary intervals 358, to provide coated needle shafts 364 having lengths ranging from 1.5 cm to 10 cm. Each needle shaft 364 has a base or first portion with a substantially uniform diameter and a second portion with a predefined taper that culminates into a tip at a first distal end 366. At step 316, for each coated needle shaft 364, the first end 366 is ground to expose the underlying conductive wire 352, to make a sharp tip and to generate a plurality of bevels on the tip to form cutting edges for skin and tissue penetration. In some embodiments, the grinding process differs from conventional needle grinders in that it uses abrasive grit on a rotating disk to cut through the extremely hard first coating.

At step 318, a second proximal end 368 of the coated needle shaft 364 is ground to expose the underlying conductive wire 352 to allow for an electrical connection of wire 352 with a lead wire 370 and assembly of a hub 372. Thereafter, at step 320, a second coating is applied to the needle shaft 364 to reduce needle insertion force. In embodiments, the second coating is of at least one lubricant such as, but not limited to, silicone oil, molybdenum disulfide or tungsten disulfide.

In accordance with some embodiments, steps 302 through 320 are directed towards fabricating a monopolar needle electrode. However, in various embodiments, the method 300 may be adapted to fabricate a variety of needles such as, for example, concentric needles, peripheral nerve block needles, subdural EEG needles, Botox injection needles and single fiber needles (a type of concentric needle with a small contact that comes out the side instead of the tip), and multi-contact needles.

Referring to FIG. 3A, to fabricate a concentric needle electrode, a first additional step 313a (after step 312 and prior to step 314) includes applying a conductive second coating or metallization layer over the first coating and a second additional step 313b after step 313a includes applying a third coating or layer of conductive but low friction material such as, but not limited to, nitrogen doped DLC over the second coating or metallization layer. Also, step 320 is not required while fabricating the concentric needle electrode.

### Method of Fabricating a Botox Injection Needle

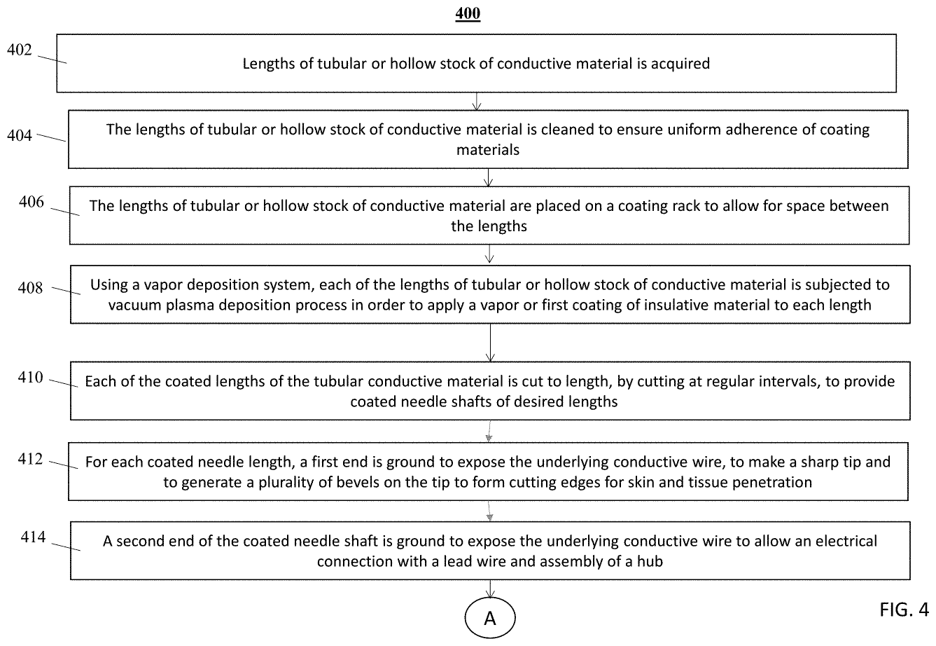

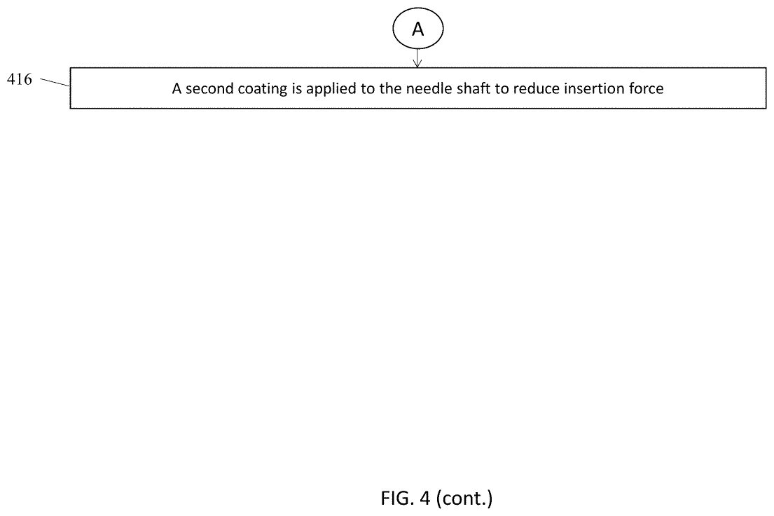

In embodiments, the methods of the present specification may be used for fabricating a Botox injection needle. Referring now to FIG. 4 which illustrates a method 400 for fabricating a Botox injection needle, instead of a stock of raw wire, at step 402, a plurality of lengths of a tubular or hollow stock of conductive material are acquired.

At step 404, each of the plurality of lengths of a tubular or hollow stock of conductive material is cleaned prior to coating, in subsequent steps, to ensure uniform adherence of coating materials.

At step 406, each of the plurality of lengths of a tubular or hollow stock of conductive material is placed on a coating rack to allow for space between the turns of each of the plurality lengths of a tubular or hollow stock of conductive material. In embodiments, the rack is sized to fit in commercially available vapor deposition equipment (for vacuum plasma deposition). The spacing is needed for vapors to reach the full circumference of each of the plurality of lengths of the tubular or hollow stock of conductive material. Each length of tubular conductive material may provide a yield ranging from 1 to 50 needle blanks.

At step 408, using a vapor deposition system (such as 362 shown in FIG. 3B), each of the plurality of lengths of the tubular conductive material is subjected to a vacuum plasma deposition process (such as, for example, an RF-based plasma-assisted chemical vapor deposition (PACVD)) in order to apply a vapor or first coating of insulative material to each length of tubular conductive material. In some embodiments, the vapor deposition process provides the first coating of a thickness of at least 20 microns. It should be appreciated that the vapor deposition process continues for 10 to 20 times the coating time needed for most prior art applications in order to achieve the at least 20 microns thick first coating. The first coating is thicker, compared to prior art applications, in order to reduce the electrical capacitance of the needle body.

In various embodiments, the electrically insulative first coating is of at least one biocompatible Diamond-like Carbon (DLC) material such as, but not limited to, Fluorinated DLC compounds having high lubricity. In some embodiments, the first coating may include one or more than one of the DLC materials applied sequentially to obtain optimal electrical, mechanical and frictional properties. In some embodiments, the first coating may include one or more than one of the following materials applied sequentially to obtain optimal electrical, mechanical and frictional properties: DLC materials, titanium nitride (TiN) coating, TiNAg which is antimicrobial, ceramic-like/epoxy/polyurethane (Polysiloxane), aluminum titanium nitride (Altin) and silver/silver chloride.

At step 410, each of the coated lengths of the tubular conductive material is cut to length, by cutting at regular intervals, to provide coated needle shafts having lengths ranging from 1.5 cm to 10 cm.

At step 412, for each coated needle length, a first end is ground to expose the underlying conductive wire, to make a sharp tip and to generate a plurality of bevels on the tip to form cutting edges for skin and tissue penetration. In some embodiments, the grinding process differs from conventional needle grinders in that it uses lapidary techniques to cut through the extremely hard first coating.

At step 414, a second end of the coated needle length is ground to expose the underlying conductive wire to allow an electrical connection with a lead wire and assembly of a hub. Thereafter, at step 416, a second coating is applied to the needle length to reduce insertion force. In embodiments, the second coating is of at least one lubricant such as, but not limited to, silicone oil, molybdenum disulfide or tungsten disulfide.

The above examples are merely illustrative of the many embodiments of the needle electrode of present specification along with associated methods of fabrication. Although only a few embodiments of the present specification have been described herein, it should be understood that the present specification might be embodied in many other specific forms without departing from the spirit or scope of the specification. Therefore, the present examples and embodiments are to be considered as illustrative and not restrictive, and the specification may be modified within the scope of the appended claims.

We claim:

1. An electrode configured for use for use in electromyography procedures, comprising:
a cylindrical shaft having a first end and a second end, wherein the cylindrical shaft consists of a conductive material;
a first coating that is electrically insulative and is configured to encase an entirety of the conductive material;
a tapered tip defined by an angled surface and positioned at the first end of the shaft, wherein the tip comprises a portion of the cylindrical shaft having a first portion of the first coating removed therefrom to thereby expose a first length of conductive material that was positioned under the removed first portion of first coating and form the angled surface; and
a hub positioned at the second end of the shaft, wherein the hub is positioned on a portion of the cylindrical shaft having a second portion of the first coating removed therefrom to thereby expose a second length of conductive material that was positioned under the removed second portion of first coating, and wherein the hub is configured to electrically couple a lead wire to the second length of conductive material at the second end.

2. The electrode of claim 1, wherein the conductive material comprises tungsten or stainless steel.

3. The electrode of claim 1, wherein the conductive material has a thickness ranging from 25 to 32 gauge.

4. The electrode of claim 1, wherein the first coating has a minimum thickness of 20 microns.

5. The electrode of claim 1, wherein the first coating comprises at least one biocompatible diamond-like carbon (DLC) material.

6. The electrode of claim 1, wherein the tapered tip comprises at least two bevels.

7. The electrode of claim 1, wherein the cylindrical shaft and tapered tip has a first portion and a second portion, wherein the first portion is of a first length and has a substantially uniform diameter, wherein the second portion is of a second length and has a tapered surface, and wherein the tip comprises the second portion.

8. The electrode of claim 7, further comprising: a second coating positioned over the first coating, wherein the second coating is at least one of molybdenum disulfide, tungsten disulfide or silicone oil.

9. The electrode of claim 7, further comprising: a conductive third coating positioned over the second coating, wherein the third coating comprises a doped diamond-like carbon material.

10. A method of fabricating an electrode in a shape of a needle and configured for use in electromyography procedures, comprising:
acquiring a conductive length of wire;
deforming the length of wire at a plurality of predetermined intervals, wherein said deforming reduces a diameter of the length of wire at each of the plurality of predetermined intervals and generates a plurality of deformations;
placing the length of wire on a support;
applying an insulative first coating to the length of wire to obtain a coated length of wire;
cutting the coated length of wire at each of the plurality of deformations to obtain a plurality of coated shafts, wherein each of the plurality of coated shafts is defined by a first end and a second end;
grinding the first end of each of the plurality of coated shafts to transform a cylindrical first end into an angled tip with at least two bevels;
grinding the second end of each of the plurality of coated shafts to remove a portion of the first coating at the second end and thereby expose a conductive length of wire under the removed portion of the first coating;
positioning a hub at the second end of each of the plurality of coated shafts;
electrically connecting a lead wire to the exposed conductive length of wire at the second end of each of the plurality of coated shafts via the hub; and
applying a second coating to each of the plurality of coated shafts and tips, wherein the second coating is different than the first coating.

11. The method of claim 11, further comprising, after deforming the length of wire at the plurality of predetermined intervals, cleaning the length of wire.

12. The method of claim 11, further comprising stretching the length of wire beyond its elastic limit.

13. The method of claim 11, wherein the insulative first coating is applied using a vacuum plasma deposition process, and wherein the vacuum plasma deposition process is repeated to achieve a thickness of the first coating of at least 20 microns.

14. The method of claim 14, wherein the thickness of the first coating reduces an electrical capacitance of the electrode.

15. The method of claim 11, wherein the first coating is a biocompatible diamond-like carbon (DLC) material.

16. The method of claim 16, wherein the second coating comprises at least one of molybdenum disulfide, tungsten disulfide or silicone oil.

17. The method of claim 11, wherein each of the plurality of coated shafts has a first portion and a second portion, wherein the first portion is of a first length and has a substantially uniform diameter, wherein the second portion is of a second length and has a tapered surface, and wherein the second portion comprises the tip at the first end of each of the plurality coated shafts.

18. The method of claim 11, wherein the length of wire comprises at least one of tungsten or stainless steel.

19. The method of claim 19, wherein the length of wire has a thickness ranging from 25 to 32 gauge.

20. A needle electrode for use in electromyography procedures, comprising:
a shaft having a first end and a second end, wherein the shaft includes a conductive core that is covered by an electrically insulative first coating followed by a second coating, wherein the first coating is of at least one DLC material, wherein the second coating is of a lubricant, and wherein the first coating has a thickness of at least 20 microns;
a tip at the first end, wherein the tip is formed by grinding the first end to expose the underlying conductive core, and wherein the tip includes at least two bevels; and
a hub positioned at the second end, wherein the hub is positioned after grinding the second end to expose the underlying conductive core, and wherein the exposed conductive core at the second end is electrically coupled to a lead wire via the hub.

21. The needle electrode of claim 20, wherein the shaft has a first portion and a second portion, wherein the first portion is of a first length and has a substantially uniform diameter, wherein the second portion is of a second length and has a tapering surface, and wherein the second portion includes the tip at the first end.
